(19) United States
(12) Patent Application Publication
(10) Pub. No.: US 2017/0020554 A1
(43) Pub. Date: Jan. 26, 2017

(54) DEVICES AND METHODS FOR PERCUTANEOUS TRANSLUMINAL ANGIOPLASTY AND ATHERECTOMY INTERVENTION PROCEDURES

(71) Applicant: TransMed7, LLC, Portola Valley, CA (US)

(72) Inventors: James W VETTER, Portola Valley, CA (US); Eugene H VETTER, Portola Valley, CA (US)

(21) Appl. No.: 15/054,667

(22) Filed: Feb. 26, 2016

Related U.S. Application Data
(60) Provisional application No. 62/126,580, filed on Feb. 28, 2015.

(51) Int. Cl. A61B 17/3207

(52) U.S. Cl. CPC A61B 17/320725

(57) ABSTRACT

A vascular interventional device may comprise a combination of inflatable and cutting elements and embodiments may comprise structures and functionality for dilatation of blood vessels narrowed by diseases, removal of plaque and debris from diseased blood vessels and delivery of drugs for the prevention of re-narrowing of vessel luminal cross-section area as well as to provide scaffolding when needed to keep vessels patent.

### BACKGROUND

Embodiments relate to medical devices and methods. More particularly, embodiments relate to vascular intervention devices including those used in percutaneous transluminal angioplasty and atherectomy procedures.

### SUMMARY

Embodiments are drawn to medical devices and methods that are used for vascular interventions. Embodiments may comprise structures and functionality for dilatation of blood vessels narrowed by diseases, removal of plaque and debris from diseased blood vessels and delivery of drugs for the prevention of re-narrowing of vessel luminal cross-section area as well as to provide scaffolding when needed to keep vessels patent. Embodiments may be portable, disposable or reusable and may be electrically, mechanically, pneumatically, hydraulically and/or manually powered and operated. The devices in this description include components that may be used together or separately to accomplish various vascular interventions in diseased vessels, where the disease encroaches on the luminal cross-sectional area of a vessel, which limits flow through the vessel or vessels, or which results in unstable or ectatic segments at risk for rupture.

### BRIEF DESCRIPTION OF THE DRAWINGS

FIG. 1 is a perspective view of a dilatation and atherectomy device assembly according to one embodiment;

FIG. 2 is a composite view of individual components of combined dilatation and atherectomy devices according to one embodiment;

FIG. 3 is a side view of a combined dilatation and atherectomy catheter assembly in the top panel and in the lower panel are two end-on views of combined dilatation and atherectomy catheter assemblies, according to one embodiment;

FIG. 4 is a perspective view of a combined dilatation and atherectomy catheter assembly according to one embodiment; and

FIG. 5 is a perspective view in two panels showing two states of use of a combined dilatation and atherectomy catheter assembly according to one embodiment.

### DETAILED DESCRIPTION

One embodiment is an interventional device, comprising separately movable components for remodeling, stabilizing and improving the lumen area of vascular structures, including dilatation of vascular walls and removal of diseased tissue elements. The separately movable components may be separately introducible designed to provide coring and part-off (atherectomy) of diseased tissues from a vascular wall. In one embodiment, the interventional device may be configured to enable distal flow while coring takes place. The device may be configured to provide directional isolation, directional pressure and depth limitation of coring.

One embodiment is an interventional tissue-removing device that is configured to provide real-time modification of area over which cording and/or tissue removal takes place, as well as to provide real-time modification of the depth of coring. The interventional tissue-removing device may be configured to deliver beneficial agents to the vascular wall and lumen to enhance vascular stability during and/or after a procedure and/or to enhance long term patency. The interventional tissue-removing device may be configured to remove whole intact cored specimens proximally into a receptacle outside the patient's body while coring proceeds. The removal of tissue by the interventional tissue-removing device may be carried out by axial oscillation and/or rotation.

One embodiment is an interventional tissue-removing device configured to part-off tissue by bearing against an independently movable and expandable structure. The interventional tissue-removing device, according to one embodiment, may include a visually apparent real-time indicator of luminal size. The interventional tissue-removing device may be configured to provide distal tissue deposition and storage through invagination of an expandable component.

One embodiment is a method of controlling an area and depth of removal of diseased tissue from a vascular wall using independently movable components.

Reference will now be made in detail to the construction and operation of embodiments illustrated in the accompanying drawings. The following description is only exemplary of the embodiments described and shown herein. The embodiments, therefore, are not limited to these implementations, but may be realized by other implementations.

Vascular wall disease, over time, can significantly thicken the walls and decrease (often asymmetrically) the cross-sectional diameter of blood vessels. This process of luminal cross-section reduction can also occur suddenly when unstable disease bulk ruptures abruptly causing thrombus formation and often, total occlusion of the affected artery, resulting in significant muscle and nerve damage and destruction in the vessel outflow areas. Percutaneous procedures such as transluminal angioplasty and transluminal atherectomy rely on increasing downstream blood flow by enlarging the cross-sectional diameter of such vessels

Many technologies have been developed to treat these areas, either to remodel the diseased walls of vessels through dilatation (transluminal angioplasty) or removal of some of the bulk (transluminal atherectomy), both of which procedures may be followed by scaffolding the walls with implanted devices that may be permanent (stents) or may be removed or dissolve themselves over time (temporary stents). These devices may also be coated with various drugs to limit re-narrowing of the vessel lumen that commonly occurs during the healing process. The limitations of the current technologies are well known and widely reported.

Embodiments overcome many of the limitations of current technologies, with particular attention to:
ease of delivery of the devices into the vascular areas of disease,
more effective treatment of the diseased areas, particularly in areas of asymmetry of disease,
more precise treatment endpoints with these devices and methods,
less trauma in treatment areas as well as areas collateral to treatment sites,
   enhanced safety at the treatment sites as well as areas distal to the diseased segments and, additionally, and improved efficiency of the procedures.

The foregoing are general capabilities of the devices and methods according to embodiment. More specific device and method capabilities will become apparent upon study of the following descriptions and figures herein.

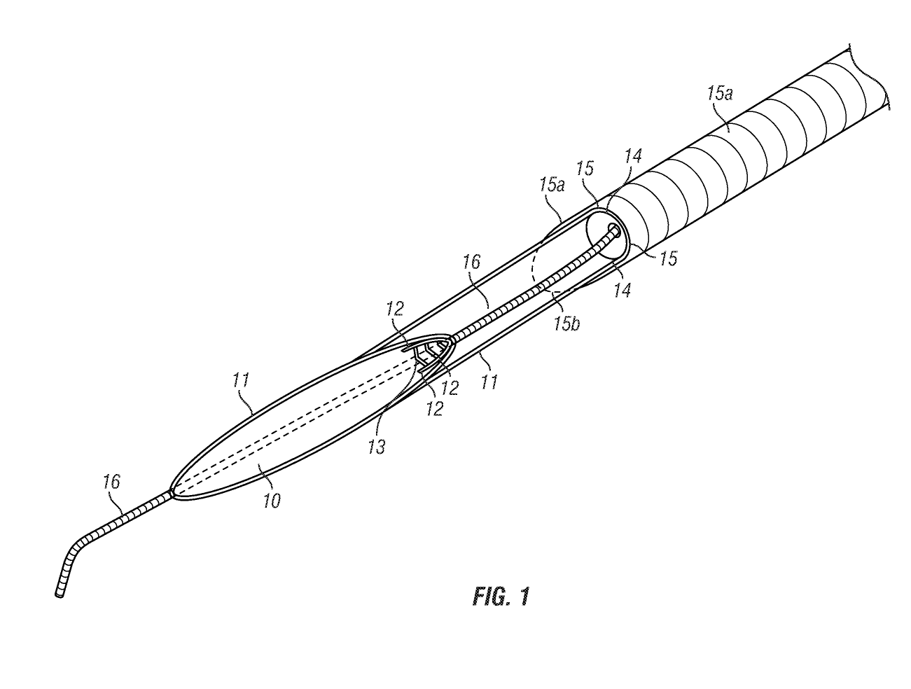

Reference will now be made in detail to the construction and operation of embodiments illustrated in the accompanying drawings. FIG. 1 shows an assembly including an inflatable (or otherwise expandable) distal component 10 located upon a scoopula 11, according to one embodiment. The scoopula 11 may be independently movable with respect to a flexible tubular housing 15a, whose distal end at 15 and 15b also may be scoopula-shaped, albeit of a slightly larger diameter relative to scoopula 11. Inflatable distal component 10 may be equipped with hardened part-off augmentation struts 12 disposed so as to surround at least a portion of the expandable distal component 10. The struts 12 may act as sheer components upon which cutter-corer 14 may bear against upon completion of its coring motions. Cutter-corer 14 may be rotatable and axially movable along the scoopula-shaped distal end 15, 15b of the flexible tubular housing 15a and may travel axially to bear against hardened struts 12 surrounding at least a portion of the expandable distal component 10 to finish severing tissue from its attachment to, for example, a vascular wall.

Scoopulae 15 and 11 may be configured to support cutter-corer 14 and inflatable component 10, respectively, while also providing directionality to diseased tissue removal. Tubular element 16 may be configured to house a coaxially-located guide element such as a shape-able wire and also may be utilized to hydraulically expand/inflatable expandable distal component 10. The location of the expandable distal component 10 is shown as being coaxially aligned with cutter-corer 14. However, it may advantageously be located entirely within scoopula 11. In this illustration, tubular element 16 is shown as a one-piece guiding and expanding tube. However, other embodiments may also include a coaxially-located and independently movable guiding element such as a shapeable wire. Tubular element 16 may also house an independently movable imaging devices, such as intravascular ultrasound instruments, flow reserve instruments and/or other devices and functionalities. Tubular element 16 may also include structures configured for the delivery of therapeutic elements, which may be eluted via weeping wire techniques as well as weeping balloon techniques to deliver pharmacologic elements directly to a vascular wall.

Part-off augmentation struts 12 may be equipped as shown with visual lumen sizing gauges that may be useful to judge proper sizing of cutter-corer instruments as well as to indicate desired luminal size endpoints during the procedure which, in the case of coronary vascular interventions, may range from about 1.5 mm to 4.5 mm or more depending on the area of treatment interest.

It is to be understood, however, that the foregoing dimensions and any dimensions referred to herein are exemplary in nature only. Those of skill in this art will recognize that other dimensions and/or configurations may be implemented, depending upon the application, and that the elements of the device could be of any length or dimension, all of which are considered within the scope of this disclosure. Furthermore, any discussion of dimensions or ranges of dimensions or physical or dynamic aspects such as flow rates or ranges of motion or time factors herein are exemplary in nature only and should not be considered to be limiting.

According to one embodiment, the entire device may be configured to be disposable or may be configured to be reusable in whole or in part. Embodiments of the present device may be electrically powered by one or more batteries and/or external power sources through a simple electrical coupling to connect to an external power supply conveniently placed, for example, in the handle or proximal end of the present device. The entire device may also be internally or externally manually powered, mechanically powered or be powered by means such as compressed air, gas or pressurized fluid. Powering the device entirely mechanically may be advantageous in areas in which the electric grid is absent, unavailable or unreliable.

One embodiment is a method of carrying out a pre-dilatation, sizing and anchoring procedure followed by a de-bulking procedure using the pictured assembly, followed by a post-de-bulking, gentle follow-up dilatation and drug delivery with inflatable component 10. This delivery can be tailored to the areas treated in a directional manner based on scoopula 11 creating a shielding element, or drug may be delivered all around via the expandable, inflatable distal element 10 and via scoopula 11. Scoopula 11 may be temporarily left in place during and after the main procedure as an effective holding scaffolding during stabilization of the vascular wall, while permitting unhindered downstream flow. Other instrumentation may be safely withdrawn including backing a guiding catheter slightly out of the vessel ostial area to permit unobstructed flow.

Another embodiment is a method of carrying out a simplified procedure where expandable distal element 10 is used to provide a part-off capability for a cutter-corer element such as cutter-corer 14 without the addition of scoopula elements in an extremely low profile configuration. In this case, a cutter-corer component may itself be scoopula-shaped. In this manner, cutter-corer 14 may, prior to coring, be delivered to the site of expandable distal element 10 and then rotated or oscillated (in that case assuming directional bias is desirable) while retracting cutter-corer element 14 proximally (axially) to permit disease bulk to project in its path upon returning distally to bear against expandable distal element 10 to separate tissue from a vascular wall for example. Expandable distal element 10 may be progressively enlarged as bulk is removed and there is a desire to remove deeper wall elements. Disease pieces thus separated from the vascular wall may be retrieved by vacuum or may simply be stored proximally in cutter-corer 14 and flexible tubular housing 15a pending removal of the particulate or whole cores via aforementioned vacuum, or alternatively, simply removing cutter-corer 14 and tubular element 15a as well as optionally expandable component 10, and if included in the assembly, scoopula (trough-shaped element) 11. The structure referenced by numeral 11 need not necessarily be scoopula shaped and, although it is shown as being sandwiched between the scoopula-shaped distal end of the flexible tubular housing 15a and cutter-corer 14, scoopula 11 may comprise a straight backbone shape or may be eliminated altogether. Scoopula 11 may also reside wholly outside of flexible tubular housing 15a and corer-cutter 14, since corer-cutter 14 need not dislodge expandable distal element 10 from scoopula 11 because its advancement may end with parting off of tissue against the proximal segment of expandable distal element 10 (and struts 12). In fact, expandable distal element 10 need not be expandable in order to be effective in its intended use, but its capabilities may be enhanced when configured as being expandable. The expandable distal element 10 may also be easier to deliver and remove if it may be expanded or contracted at will and it may also be useful as a distal anchor to aid in the delivery and removal of other elements of the assembly.

It is to be understood that the above descriptions are but exemplary methodologies and that one or more of the steps described above may be omitted, while other steps may be added thereto to any of these embodiments, depending on the target site within the body. The order of some of the steps may additionally be changed, according to the desired procedure.

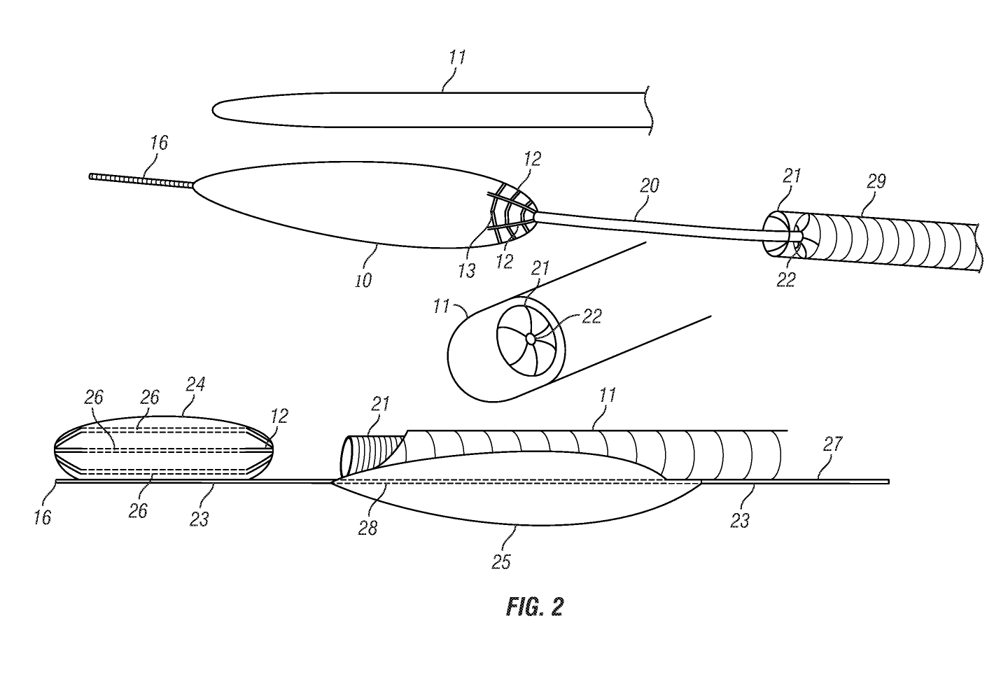

FIG. 2 shows a compilation of components that may be a part of an assembly or may be utilized separately depending on the nature and goals of an interventional procedure. Scoopula 11 is shown as it may exist in simple form, however it may also contain an attachment configuration to expandable distal element 10 that enables it to deliver expandable distal element 10 together with itself distally to a vascular location. Scoopula 11 may contain a guiding wire lumen in the event a movable guiding wire is desirable. Scoopula 11 may itself terminate in a shapeable fixed guiding wire element. Expandable distal element 10 is shown in FIG. 2 delivered over a tubular element 16 comprising a guiding wire that may define a coaxial space configured for injection of contrast or pharmacologic agents downstream. Also shown is an axle element 20 over which cutter-corer 21 and cutter-corer 22 may be advanced to bear against part-off struts 12. Shown as well are visual size gauge elements 13. In this example, driving tube 29 is shown with helical elements that are configured to impart flexibility and that may also be used to enhance transport of tissue removed from a vascular wall and lumen.

In a perspective view, cutter-corer component 21 and cutter-corer 22 are shown in a configuration that may serve to macerate tissue as it is removed, or may serve to enhance stacking against expandable distal element 10 for later removal. Expandable distal element 10 may itself contain spaces between part-off struts 12 where such tissues may be stored for later removal with the withdrawal of expandable distal element 10 from the vessel. If expandable distal element 10 is a balloon type structure, these spaces may be segregated from the fluid-tight areas of the expandable section(s).

The lower panel of FIG. 2 shows an embodiment that includes additional configurations of expandable element 10, such as expander 24 to distinguish its attachment to being wholly deliverable and expandable vial introducer 23, which in this case involves hydraulic expansion of expander 24 via a fluid connection with introducer 23. Expandable distal element 10 may also include part-off struts 12 extending along the length of expander 24 in order to constitute removable or permanent scaffolding for the vessel lumen during and optionally after the procedure.

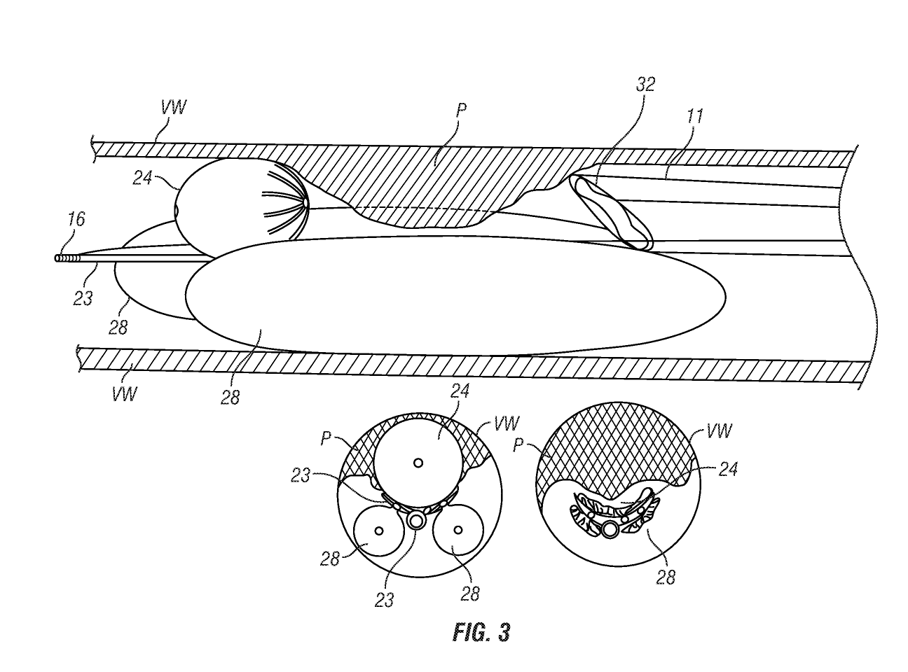

An additional expander 25 is shown as an expandable supporting structure for creating enhanced directional bias where disease asymmetry is encountered. In this case, through-lumen side bias pressure stabilizer(s) 28 (see FIG. 3) may be provided to permit independent movement of expander 25 with respect to the scoopula-terminated flexible tubular housing 15a as well as supporting introducer 23 and its attached expander 24. In this manner, capability may be added that permits support in precisely the location desired for optimized coring direction and length.

The lower panel of FIG. 2 shows aspects of a method that may be used to define an area of isolation of a treatment segment. Indeed, an area of treatment may be defined by altering the distance between the starting point of cutter-corer 21 and its endpoint against the part-off struts 12, utilizing the independent movement capabilities provided by introducer 23/expander 24, flexible tubular housing 15/cutter-corer 21 and 27/expander 25. Coupling an area of treatment with a depth defined by the expanded size of expander 24 (based on hydraulic pressure input for example) together with bias forces provided by expander 25 (again, based on hydraulic pressure input for example), precise control may be achieved of the amount and depth of diseased tissue removed from a vascular wall for example. In this case, expander 24 may serve as an effective depth limiter to prevent undesirable coring into deep-wall components.

FIG. 3 illustrates details of a vascular wall disease scenario with expander 28 and expander 24 on their introducer 23, which may also contain a tubular element 16 comprising a separate wire or catheter in place such that corer-cutter 32 may be advanced together with or independently from scoopula 11 to shave off diseased tissue “P” (labeled “P” for plaque, designating that in this example, the disease comprises mainly atheromatous plaque) from the vessel wall (“VW”) to a depth as limited by expander 24 and side bias pressure stabilizer 28. The arrangement of these various components is further illustrated in the cross-sectional drawings in FIG. 3.

In the lower left panel, expander 24 and side bias pressure stabilizer 28 are shown as full diameter, defined as fully occupying the available internal diameter of an exemplary vessel cross-sectional lumen. In the panel on the left, the plaque P is partially obscured by the distal placement of expander 24, given that this view of an exemplary vessel is viewed from a distal vantage point looking back in a proximal direction. The left-hand lower panel also shows that the use of two or more side bias pressure stabilizer 28, which provide spaces permitting flow along the assembly and into the distal vascular bed. This provision significantly enhances safety of the entire procedure by permitting calm time within which to proceed with precision removal of disease, such that deep-wall components of the vessel wall VW remain unharmed.

In the lower right panel of FIG. 3, expander 24 and side bias pressure stabilizer element 28 are shown as being of the expandable variety and in their non-expanded configuration. This panel also illustrates the exceedingly common occurrence of asymmetric plaque P burden, as well as the low profile achievable with expandable components that may be more easily advanced into correct position prior to advancing potentially bulkier components of a system for coring and retrieval of diseased vascular elements. These panels taken together also illustrate that maintenance of blood flow throughout a procedure may be realized, as a result of the initial low profile of introducer 23 and its expander 24 and side bias pressure stabilizer element 28.

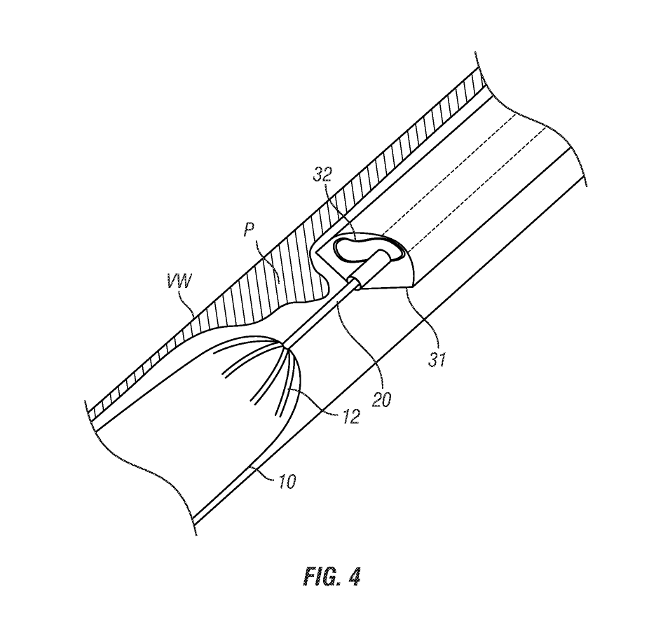

FIG. 4 illustrates a perspective view of components of an assembly comprising inflatable distal component 10 equipped with part-off enhancing struts 12, a supporting axle 20 that may coaxially provide an inflation lumen as well as a guiding element lumen, together with a crescent shaped, low-profile cutter-corer component whose corer-cutter 32 may oscillate rotationally and axially to provide cutting efficiency in a variety of tissue compositions. The distal edge of delivery/transport element 31 may be optimized to pass easily through constricted areas of a vascular space (bounded between the proximal end of the expandable distal component 10 and the distal end of the cutter-corer 32, as shown in FIG. 4) and its tubular portion may be highly flexible in order to follow supporting axle 20 (that may also function as a guiding element) through the vascular spaces. Distal axial exposure of corer-cutter 32 may be controlled to provide safety while limiting depth of coring. The vascular wall VW is shown together with its thickening, typically asymmetrically disposed disease plaque.

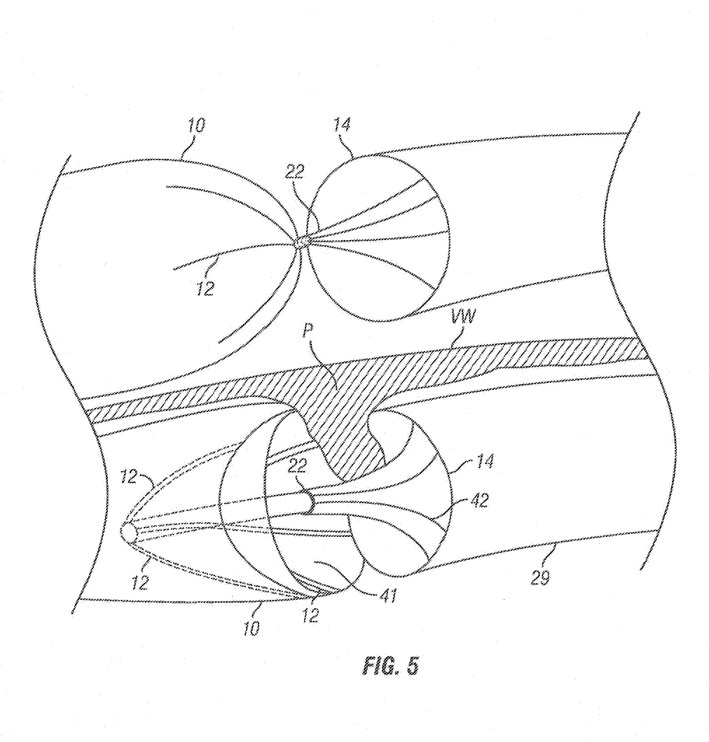

FIG. 5 illustrates expandable distal component 10 and struts 12 in an embodiment that provides invagination capability for deposition and storage of plaque P elements removed from the vascular wall VW into a volume indicated by 41, which volume is created and preserved singly or in combination by the inversion of expandable distal component 10 and struts 12 together with, for example, a ratcheting element or other resistance mechanism built in to an axle over which cutter-corer 22 and inner surface of expandable distal component 10 rides. A pressure relieving element may also be provided that may be actuated by cutter-corer 22, or that may be passively actuated in response to pressure exerted by cutter-corer 22, that because fluid is in this way released, expandable distal element 10 would tend not to reoccupy its original (greater) more spherical volume. In this embodiment, cutter-corer 22 of cutter-corer 14 provides axial forces to invaginate expandable distal component 10 together with its part-off struts 12.

In FIG. 5 macerating elements 42 of cutter-corer 14 may be configured to macerate diseased tissue removed from, for example, a vascular wall or may be more advantageously utilized to wrap and stack the diseased cores in an orderly manner against the inner invaginated wall of expandable distal component 10, as shown in the lower panel.

Embodiments may be formed of or comprise one or more biocompatible materials such as, for example, stainless steel or other biocompatible alloys, and may be made of, comprise or be coated with polymers, such as polyimide, and/or biopolymer materials as needed to optimize function(s). Some of the components may be purposely surface-treated differentially with respect to adjacent components, as detailed. The various gears or pulleys that may be used in driving mechanisms of the various elements described herein may be made of any suitable, commercially available materials such as nylons, polymers such as moldable plastics, and others. If used, the motor powering the various powered functions of the present biopsy device may be a commercially available electric DC motor. The handle of the present device, although not shown herein but implied in method descriptions may likewise be made of or comprise inexpensive, injection-molded plastic or other suitable rigid, easily hand held strong and light-weight material. The handle may be configured in such a way as to make it easily adaptable to one of any number of existing guiding platforms, such as stereotactic table stages. The materials used in the present biopsy device may also be carefully selected from a ferro-magnetic standpoint, such that the present biopsy device maintains compatibility with MRI equipment.

The power source may comprise an external commercially available AC to DC transformer approved for medical device use and plugged into the provided socket in the present biopsy device, or may comprise an enclosed battery of any suitable and commercially available power source. The battery, if used, may be of the one-time use disposable (and optionally recyclable) variety, or may be of the rechargeable variety. Additionally, other power sources, for example, mechanical linkages or compressed air or fluid motors, may be used.

While certain embodiments of the disclosure have been described, these embodiments have been presented by way of example only, and are not intended to limit the scope of the disclosure. Indeed, the novel methods, devices and systems described herein may be embodied in a variety of other forms and other applications. All such other applications making use of the principles disclosed herein for this device and that could be envisioned by one skilled in the art are therefore considered to be within the scope of this disclosure. Furthermore, various omissions, substitutions and changes in the form of the methods and systems described herein may be made without departing from the spirit of the disclosure. The accompanying claims and their equivalents are intended to cover such forms or modifications as would fall within the scope and spirit of the disclosure. For example, those skilled in the art will appreciate that in various embodiments, the actual physical and logical structures and dimensions thereof may differ from those shown in the figures. Depending on the embodiment, certain steps described in the example above may be removed, others may be added. Also, the features and attributes of the specific embodiments disclosed above may be combined in different ways to form additional embodiments, all of which fall within the scope of the present disclosure. Although the present disclosure provides certain preferred embodiments and applications, other embodiments that are apparent to those of ordinary skill in the art, including embodiments which do not provide all of the features and advantages set forth herein, are also within the scope of this disclosure. Accordingly, the scope of the present disclosure is intended to be defined only by reference to the appended claims.

What is claimed is:

1. A device, comprising:
an independently-movable distal expandable element configured to be introduced and advanced into vasculature in a contracted state, and to be anchored, in an expanded state, within the vasculature at a position distal to tissue to be cut;
a plurality of reinforcing struts disposed against and around at least a proximal portion of the independently-movable distal expandable element; and
an independently movable tissue cutting assembly configured for introduction and axial advancement to a selectable position, in the vasculature, proximal to the tissue to be cut,
wherein the tissue cutting assembly is configured to advance within the vasculature toward the distal expandable element while cutting tissue and parting off the cut tissue from surrounding tissue as the tissue cutting assembly is caused to bear against at least some of the plurality of reinforcing struts.

2. The device of claim 1, wherein the independently-movable distal expandable element is inflatable.

3. The device of 1, further comprising a tubular element disposed within and through at least one of the independently-movable distal expandable element and the independently movable tissue cutting assembly.

4. The device of claim 1, wherein an area over which diseased tissue is to be cut is selectable by selectably positioning the independently-movable distal expandable element and the independently movable tissue cutting assembly within the vasculature.

5. The device of claim 1, further including at least one of a first scoopula and a second scoopula, configured to assist in a positioning of the independently-movable distal expandable element and the independently movable tissue cutting assembly, respectively.

6. The device of claim 1, further including at least one expandable bias stabilizer, configured to bias the independently movable tissue cutting assembly against the tissue to be cut within the vasculature.

7. The device of claim 1, wherein the reinforcing struts comprise a visually apparent real-time indicator of luminal size.

8. The device of claim 1, wherein the independently-movable distal expandable element is configured to be advanced within the vasculature and positioned before the independently movable tissue cutting assembly.

9. The device of claim 1 wherein the independently-movable distal expandable element is configured to be expanded such that an outer surface thereof presses against an interior vascular wall.

10. The device of claim 1, wherein the distal expandable element and the tissue cutting assembly are each independently movable to enable the tissue cutting assembly to sequentially cut tissue in different areas of the vasculature.

11. A method, comprising:
introducing and advancing an independently-movable distal expandable element to a first operator-selectable position, in the vasculature, that is distal to tissue to be cut;
introducing and advancing an independently movable tissue cutting assembly to a second operator-selectable position, in the vasculature, that is proximal to the tissue to be cut;
expanding and anchoring the independently-movable distal expandable element against an internal wall of the vasculature;
advancing the independently movable tissue cutting assembly toward the independently-movable distal expandable element while cutting tissue; and
parting off the cut tissue by causing the independently movable tissue cutting assembly to bear against a proximal portion of the independently-movable distal expandable element.

12. The method of claim 11, wherein the independently-movable distal expandable element comprises a plurality of reinforcing struts disposed against and around at least a proximal portion thereof and wherein parting off is carried out with the independently movable tissue cutting assembly being caused to bear against at least some of the plurality of reinforcing struts.

13. The method of claim 11, wherein an area in which tissue is to be cut is operator-selectable, through selection of at least one of the first and second operator-selectable positions.

14. The method of claim 11, further comprising determining a luminal size of the vasculature into which the independently movable tissue cutting assembly is inserted through observation of a state of the reinforcing struts disposed against and around the expanded and anchored independently-movable distal expandable element.

15. The method of claim 11, further comprising moving at least the independently-movable distal expandable element to a third operator-selectable position, in the vasculature, to thereby enable the independently movable tissue cutting assembly to cut and part-off new tissue.

16. The method of claim 11, wherein the independently-movable distal expandable element and the independently movable tissue cutting assembly are introduced and advanced in the vasculature separately and independently of one another.

17. The method of claim 11, further comprising selectably biasing the independently movable tissue cutting assembly against the tissue to be cut by expanding at least one expandable bias stabilizer within the vasculature.

18. The method of claim 11, wherein expanding and anchoring the independently-movable distal expandable element causes the independently-movable distal expandable element to press against an interior vascular wall.

19. The method of claim 11, further comprising supporting the independently movable tissue cutting assembly using a scoopula-shaped distal end of a tubular member.
